(19) United States
(12) Patent Application Publication
(10) Pub. No.: US 2020/0268297 A1
(43) Pub. Date: Aug. 27, 2020

(54) Auscultare

(71) Applicant: Crystal Sylver, San Francisco, CA (US)

(72) Inventor: Crystal Sylver, San Francisco, CA (US)

(21) Appl. No.: 16/283,758

(22) Filed: Feb. 23, 2019

(51) Int. Cl. A61B 5/16; A61B 8/02; A61B 7/04

(52) U.S. Cl. CPC A61B 5/164; A61B 5/0255; A61B 7/04; A61B 8/02

(57) ABSTRACT

The invention is a lie detector for the heart.

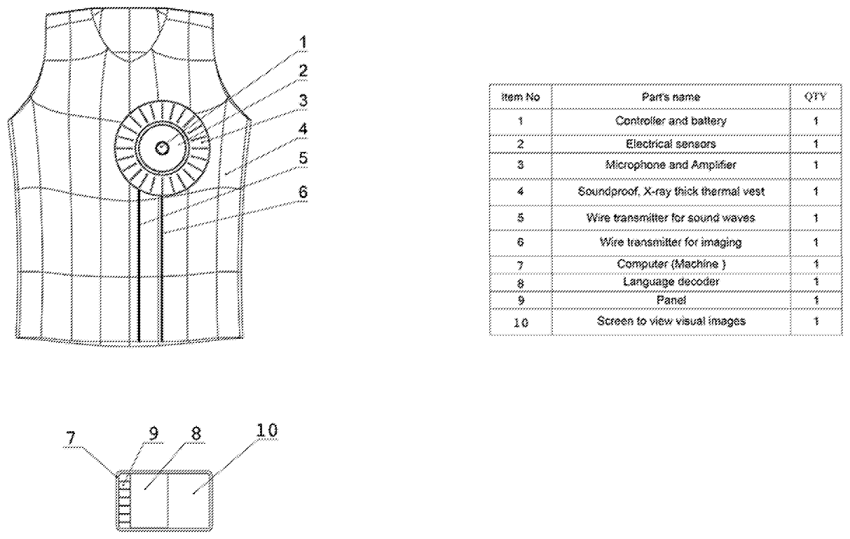

### CROSS-REFERENCE TO RELATED APPLICATIONS

Auscultare

Provisional Patent Filed Feb. 23, 2018

Application # 62/710,665

### STATEMENT REGARDING FEDERALLY SPONSORED RESEARCH OR DEVELOPMENT

Not Applicable

### REFERENCE TO SEQUENCE LISTING, A TABLE, OR A COMPUTER PROGRAM LISTING COMPACT DISC APPENDIX

Not Applicable

### BACKGROUND OF INVENTION

The Auscultare is a mechanical device. It is a lie detector that utilizes subsonic and infrasound electromagnetic energy frequencies to capture low frequency sound waves from the heart, where all memory is retained.

The traditional lie detector, the polygraph, only detects changes in heart rate, blood pressure, perspiration, and body movements through interrogation.

There is a growing interest in alternative techniques and technologies surrounding polygraph tests due to the inaccuracy of the test.

### BRIEF SUMMARY OF INVENTION

The Auscultare uses subsonic, infrasound, and ultrasound frequencies to capture information emitted by the heart. A computer receives and decodes the frequencies and displays the information on a monitor.

The heart muscle is a powerful and intelligent sensory organ capable of encoding and processing information and making decisions independently from the brain. The heart is the most powerful source of electromagnetic energy in the human body, generating 60 times more electrical activity in amplitude than the brain. The heart transmits emotional information via the electromagnetic field into the environment, which can be detected and processed by others. The heart encodes and transmits messages between heartbeats through waves of energy.

The memory retained by the heart is fact-based. The brain is full of delusions, lies, stories, and disorders, making interrogation with a polygraph an inaccurate method of lie detecting.

### BRIEF DESCRIPTION OF THE SEVERAL VIEWS OF THE DRAWING

1/1 Auscultare—front view of vest and computer

1. Controller and Battery

2. Electrical Sensors

3. Microphone and Amplifier

4. Soundproof, X-ray Thick Thermal Vest

5. Wire Transmitter for Sound Waves

6. Wire Transmitter for Imaging

7. Computer (Machine)

8. Language Decoder

9. Panel

10. Screen to View Visual Images

(1) Controller and Battery located in the center of the Microphone in the vest. (2) Electrical Sensors located around the Conroller and Battery. (3) Microphone and Amplifier surrounding the Electrical Sensors. (4) Soundproof, X-ray Thick Thermal Vest. (5) Wire Transmitter for Sound Waves from the Microphone down the front of the vest. (6) Wire Transmitter for Imaging from the Microphone down the front of the vest. (7) Computer (Machine). (8) Language Decoder in Computer. (9) Panel in Computer. (10) Screen to View Visual Images in the Computer.

### DETAILED DESCRIPTION OF THE INVENTION

The Auscultare incorporates a thick soundproof, x-ray thick thermal vest that wraps around the upper torso and chest. The vest is infused with foil on the inside in order to isolate electromagnetic radiation. The vest has a microphone/amplifier and electrical sensors, with controller and battery located over the heart muscle and fused into the vest. The electrical sensors attach to the chest of the individual wearing the vest. There may be more sensors placed on the chest. The controller allows different frequencies to be picked up from radio waves, sound waves, infrasound, subsonic sound, ultrasound images, and more, emitted by the heart. The information is received by the electrical sensors and microphone/amplifier and is wirelessly transmitted to the computer (machine) where the information is decoded, analyzed, reviewed. The computer allows for language decoding and a screen to view images that may be embedded in the heart.

The Auscultare is different from a polygraph lie detector test because the Auscultare uses subsonic, infrasound, and ultrasound frequencies to capture information, while a polygraph detects changes in heart rate, blood pressure, perspiration, and body movements through interrogation. The Auscultare uses a soundproof thermal vest to cover the chest and heart area with a subsonic microphone/amplifier with electrical sensors, which capture radio waves, sound waves, infrasound, subsonic sound, and ultrasound images. A computer receives and decodes the information in the energy/electrons of the sound waves, which may include DNA analysis of the sound wave and visual images retained as memory in the heart. A language decoder translates any language and transcribes the sound emitted from the heart.

The heart muscle is a powerful and intelligent sensory organ capable of encoding and processing information and making decisions independently from the brain. It acts as its own brain and retains all memory from birth. The brain is capable of multi-dimensional storytelling, lying, delusions, dementia, fragmented memory, schizophrenia, and other mental disturbances, which the heart is not. The heart retains memory in the form of fact, not stories, dreams, or delusions like the brain. The ability to process and decode the heart's memory will create a lie detector that can't be tampered with or beaten.

The electromagnetic energy field emanating from the heart at all times is the largest energy field in the body. It is over 60 times greater in amplitude than the electromagnetic field of the brain.

Today lie detector tests, known as polygraph tests, are widely used in the United States, and in a few other countries, like Canada, Japan, and Israel. They are mainly used for three reasons, investigations of specific events and criminal investigations, screening of employees, especially in security sensitive occupations, and in law enforcement agencies.

The Auscultare offers a solution to the traditional polygraph test, which has been under a great deal of scientific and public controversy in the US regarding the validity and reliability of the test, and the method by which it's given. The Auscultare will eliminate false accusations and forced confessions.

The polygraph is especially used in government sensitive occupations and in all law enforcement agencies in the US. There are an estimated 500000 polygraphs in the US alone. The US Department of Energy Polygraph Program, established in 1999, mandates polygraph examinations for several thousand employees and potential employees a year.

There is a growing interest in alternative techniques and technologies surrounding the polygraph. Those that measure and record electrical brain activity using modern functioning imaging techniques, such as the positron emission tomography (PET) and magnetic resonance imaging (MRI and fMRI) have recently received a lot of attention.

The recording and storing of thoughts, dreams, and memories for future playback is now being offered as a service by the US neurotechnology startup, Millennium Magnetic Technologies (MMT), using magnetic resonance imaging (MRI) scans. Dr. Donald Marks, the founder of MMT, has a patent pending for Cognitive Engram Technology, which can record detailed brain patterns and correlate them to thoughts and emotions for future interpretation. Those inquiring about this technology include academic institutions, like MIT, individuals, and law firms interested in the recordings for lie detection purposes. Scientists in Japan at the Computational Neuroscience Lab are also now using MRI brain scans to record dreams and reconstruct them in order to play back.

The heart, compared to the brain, is the most powerful source of electromagnetic energy in the human body, generating 60 times more electrical activity in amplitude than the brain. The magnetic field produced by the heart is more than 100 times greater in strength than the field generated by the brain and can be detected up to 3 feet away in all directions using SQUID-based magnetometers, a superconducting quantum interference device. The heart has its own consciousness and ‘brain’ and communicates energetically, which is referred to as cardioelectromagnetic communication.

Through extensive research, scientists as the HeartMath Institute in California have proposed that the rhythmic patterns emitted from the heart can transmit emotional information via the electromagnetic field into the environment, which can be detected by others and processed. They have also determined that the heart encodes and transmits messages between heartbeats through waves of energy. The HeartMath Institute has published a large body of scientific research in publications such as the Harvard Business Review and the American Journal of Cardiology, with a client list that includes companies such as Hewlett Packard, Shell, Cisco Systems, and Boeing.

The Auscultare offers a new way of detecting deep down and even hidden emotions, thoughts, desires, and memories of events, through the encoded information that the heart transmits on a subsonic level. Unlike the brain, the heart cannot tell stories or create delusions, which is very common during the interrogation process of determining whether a person is lying or not, which is a large part of the polygraph method.

The Cognitive Engram Technology that records dreams, thoughts, and memories, also just focuses on the electrical activity of the brain. The brain has too high of a capacity for memory loss, hallucinations, delusions, storytelling, and inaccurate memory recall, for it to be a reliable source of lie detection. The heart is the only reliable method of detecting lies. There have been many books published recently on the remarkable intelligence and power of the heart, including several from the HeartMath Institute.

The Auscultare may incorporate DNA analysis of sound waves to ensure the accuracy of the information being captured and recorded.

The Auscultare may also include sweat analysis.

The Auscultare may be used in the health care industry to determine the mental and physical health of an individual.

The Auscultare may be used in the education system, therapy sessions, or in any application where the truth must be determined, related to an individuals mental and physical health.

1. The invention claimed is the Auscultare comprised of:
a soundproof, X-ray thick, thermal vest that wraps around the upper torso;
a subsonic microphone/amplifier with electrical sensors, controller and battery, which captures radio waves, sound waves, infrasound, subsonic sound, and ultrasound images located over the heart and part of the vest;
wire transmitter for sound waves and wire transmitter for imaging to view on computer (machine);
a computer (machine) with panel, language decoder, and screen to view visual images. Dependent Claims

2. The Auscultare as recited in claim 1, may use a vest shaped in various ways and made of different materials to best eliminate outside frequencies.

3. The Auscultare as recited in claim 1, may use wire transmitters in any location on vest to send information and images.

4. The Auscultare as recited in claim 1, may use a larger, smaller, or different shaped microphone/amplifier and electrical sensors.

5. The Auscultare as recited in claim 1, may or may not use visual imaging from ultrasound frequencies.

6. The Auscultare as recited in claim 1, may or may not use a language decoder.

7. The Auscultare as recited in claim 1, may incorporate DNA analysis of sound waves.

8. The Auscultare as recited in claim 1, may use any number of additional electrical sensors.

9. The Auscultare as recited in claim 1, may or may not use foil on the inside of the vest.

10. The Auscultare as recited in claim 1, may or may not use sweat analysis.

11. The Auscultare as recited in claim 1, may transmit information wirelessly or through an electrical cord from the vest to the computer (machine).

12. The Auscultare as recited in claim 1, may use a combination of radio waves, sound waves, infrasound, subsonic sound, and ultrasound, plus other frequencies.
